(19) United States
(12) Patent Application Publication
(10) Pub. No.: US 2025/0152065 A1
(43) Pub. Date: May 15, 2025

(54) ACTIVE BIOSENSING ELECTRODE AND METHOD

(71) Applicant: GEORGIA TECH RESEARCH CORPORATION, Atlanta, GA (US)

(72) Inventors: John TARASIDIS, Atlanta, GA (US); Jennifer E. HASLER, Atlanta, GA (US)

(21) Appl. No.: 18/832,412

(22) PCT Filed: Feb. 1, 2023
(86) PCT No.: PCT/US2023/012088

§ 371 Date: Jul. 23, 2024

Related U.S. Application Data
(60) Provisional application No. 63/305,364, filed on Feb. 1, 2022.

(51) Int. Cl. A61B 5/277; A61B 5/256; A61B 5/257; A61B 5/263; A61B 5/28; A61B 5/291; A61B 5/296; A61B 5/302

(52) U.S. Cl. CPC A61B 5/277; A61B 5/256; A61B 5/257; A61B 5/263; A61B 5/28; A61B 5/291; A61B 5/296; A61B 5/302; A61B 2562/0214; A61B 2562/164

(57) ABSTRACT

An electrode device provides non-invasive non-contact measurements of biological signals such as from the heart, muscles, or brain (ECG, EMG, EEG) from a subject. The exemplary electrode device is a non-contact sensor that does not make an ohmic/galvanic connection to the skin but rather acquires the signal by forming a capacitor with the skin at the sensing site. The exemplary electrode device is configured with a feedback path that is defined based on, or as a function, of proximity. Thus, in response to motion, unwanted currents are cancelled, and the gain does not change.

### CROSS-REFERENCE TO RELATED APPLICATIONS

This application claims the priority benefit of U.S. Provisional Patent Application Ser. No. 63/305,364, filed Feb. 1, 2022, entitled “Active Biosensing Electrode Design,” which application is hereby incorporated by this reference in its entirety.

### BACKGROUND

### Field

Embodiments of the present invention relate to non-contact biosensor design, specifically a biosensor with a circuit designed to compensate for the motion of the sensor to reduce signal noise in the sensor output.

### Background

Non-contact sensors generally suffer from signal degradation from motion (defined as changes in proximity between the sensor and the skin). When motion occurs, the gain of the amplifier changes, and unwanted currents are injected at the input of the amplifier that can cause large, unwanted voltage swings at the output.

### BRIEF SUMMARY OF THE DISCLOSURE

Accordingly, the present invention is directed to an active biosensing electrode and method that obviates one or more of the problems due to limitations and disadvantages of the related art.

An electrode device is disclosed that can provide non-invasive non-contact measurements of biological signals such as from the heart, muscles, or brain (ECG, EMG, EEG) from a subject. The exemplary electrode device is a non-contact sensor that does not make an ohmic/galvanic connection to the skin but rather acquires the signal by forming a capacitor with the skin at the sensing site. The exemplary electrode device is configured with a feedback path that is defined based on, or as a function, of proximity. Thus, in response to motion, unwanted currents are cancelled, and the gain does not change.

In accordance with the purpose(s) of this invention, as embodied and broadly described herein, this invention, in one aspect, relates to a sensor device according to principles described herein may include an amplifier having a first input terminal, a second input terminal, and an output; a dielectric body; a capacitive sensor disposed on the dielectric body and having an output coupled to the first input terminal of the amplifier; a feedback capacitor having a first capacitive plate coupled to the output of the capacitive sensor and the first input terminal of the amplifier and having a second capacitive plate coupled to the output of the amplifier.

In another aspect, the invention relates to a biosensor according to principles described herein may include a flexible substrate; a first feedback capacitive plate on the flexible substrate; a second feedback capacitive plate on the flexible substrate; a sensor capacitive plate on the flexible substrate; and a biasing circuit connected between the second feedback capacitive plate, and the first feedback capacitive plate, wherein the first feedback capacitive plate is coupled to the sensor capacitive plate.

Additional advantages of the invention will be set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and is not restrictive of the invention, as claimed.

Further embodiments, features, and advantages of the active sensor device as well as the structure and operation of the various embodiments of the active sensor device, are described in detail below with reference to the accompanying drawings.

It is to be understood that both the foregoing general description and the following detailed description are exemplary and explanatory only and are not restrictive of the invention as claimed.

### BRIEF DESCRIPTION OF THE DRAWINGS

The accompanying figures, which are incorporated herein and form part of the specification, illustrate a sensor circuit Together with the description, the figures further serve to explain the principles of the sensor circuit described herein and thereby enable a person skilled in the pertinent art to make and use the sensor circuit.

FIG. 1 is a high-level diagram illustrating an exemplary circuit according to the principles described herein.

FIG. 2A shows an example capacitive sensor device according to the principles described herein.

FIG. 2B is a circuit diagram representative of the example capacitive sensor of FIG. 2A.

FIG. 2C is a photograph of a prototype sensor according to the principles described herein and represented by FIGS. 2A and 2B.

FIG. 3A shows an alternate design of a sensor according to the principles described herein.

FIG. 3B shows an example construction of a sensor according to the principles described herein.

FIGS. 4A and 4B show simulations (FIG. 4A) and experiment results (FIG. 4B) of the prototype system. The simulation circuit is shown in FIG. 4C.

FIG. 5 shows an example motion artifact that may be picked up by the exemplary electrode device when only the sensing plate is employed.

FIG. 6 is a schematic for an example capacitive electrode preamplifier according to the principles described herein.

FIG. 7 illustrates a capacitive level board stack-up.

FIG. 8 is a photograph showing a prototype capacitive biosensing electrode according to the principles described herein.

FIG. 9 illustrates mechanical structure according to the principles described herein.

FIG. 10 shows a simple circuit structure according to the principles described herein.

FIG. 11 illustrates an alternative circuit for implementation as a biosensor according to the principles described herein.

FIG. 12 illustrates another embodiment incorporating the basic sensing circuit described herein.

FIG. 13 illustrates another embodiment incorporating the basic biosensing circuit described herein with improved common mode rejection (CMR).

### DETAILED DESCRIPTION

Reference will now be made in detail to embodiments of the active sensor design with reference to the accompanying figures. The same reference numbers in different drawings may identify the same or similar elements.

An exemplary circuit design is disclosed for non-invasive non-contact measurements of biological signals (“biosignals”) such as from the heart, muscles, or brain (ECG, EMG, EEG) from a subject. The exemplary device is a non-contact sensor that does not make an ohmic/galvanic connection to the skin, but rather acquires the signal by forming a capacitor with the skin at the sensing site. The exemplary device is configured with a feedback path that is defined based on, or as a function, of proximity. Thus, in response to motion, unwanted currents are cancelled, and the gain does not change/is stabilized.

Because of its capacitive sensing mechanism, the exemplary device can be configured to be worn and provide robust measurements for extended periods, e.g., through clothing or in helmets. In contrast, in typical bio-measurement applications, electrodes are placed with security to avoid motion corruption (for example, with adhesive or an electrolyte gel). This can make the installation of such devices uncomfortable and render long-term measurements infeasible for patients. In addition, because the intrinsic structure of the exemplary electrode device can cancel motion corruption or artifacts, the exemplary electrode device can be employed with ultra-low-power sensor systems that do not require extensive digital signal processing or post-processing for artifact removal.

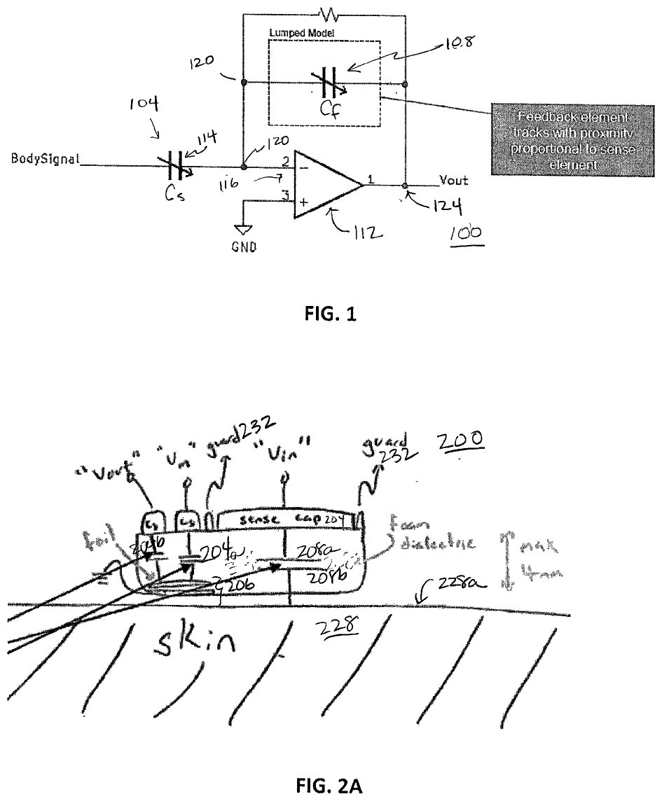

FIG. 1 is a high-level diagram illustrating an exemplary circuit 100 according to the principles described herein. The exemplary device is configured with a common charge mode amplifier topology having an input “sense” capacitor 104 (Cs) and a “feedback” capacitor 108 (Cf). The gain for this topology can be defined as Cs/Cf. By defining both the sensing capacitor Cs 104 and feedback capacitor Cf 108 based on, or as a function of, the proximity of the device to the skin, the proximity can be canceled, and thus the gain from the structure of the exemplary electrode device can be defined by the relative plate areas of a feedback plate and a sensing plate (As/Af). By designing a structure where both the feedback capacitor and the sensing capacitor track together with proximity, the exemplary electrode device can address technical implementation challenges associated ohmic contact, gain degradation, and corruption from motion.

A prototype has been developed and tested with success. In the prototype system, the amplifier 112 is configured to be actively biasing the feedback capacitor 108 with a negative potential to substantiate an equal but opposite charge to the input plate 114 of the feedback plate connected to the input of the amplifier 112. To this end, when the electrode moves, the changes in capacitance allows these charges to be released from the plates resulting in an unwanted current. But, there are equal and opposite amounts of charge at this node 120, and because both the feedback and sensing capacitors 104 and 108 both change with movement, this movement of charge-resulting cancels intrinsically. If the feedback capacitor 108 was fixed, only the charge would move from the sensing capacitor 104, and the output voltage Vout at node 124 of the amplifier would change to cancel this (presenting a large motion artifact). The exemplary electrode device does not require this change in voltage, and thus no motion artifact is induced at the output.

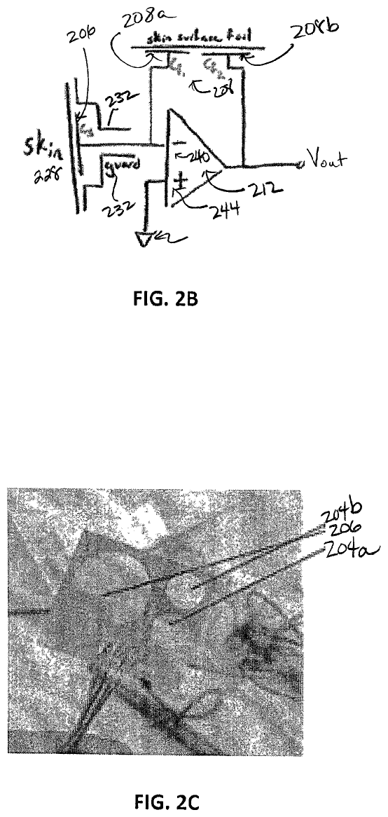

FIG. 2A shows an example capacitive sensor device according to the principles described herein. FIG. 2B is a circuit diagram representative of the example capacitive sensor of FIG. 2A. FIG. 2C is a photograph of a prototype sensor according to the principles described herein and represented by FIGS. 2A and 2B.

In the example shown in FIGS. 2A-2C, the device 200 includes a sensing capacitor 204, Cs, created by a conductive plate/capacitive sensing plate 206 in the structure that serves as one plate of the sensing capacitor 204 and the patient's body 228 that serves as the other plate in the sensing capacitor 204. There is a feedback capacitor 208, C_{f}, created by a conductive plate 208a in the structure in-plane with the capacitive sensing plate 206, and a conductive plate 208b in the structure in-plane with the body. Proximity is defined by the distance between the body surface plane 228a and the capacitive sensing plate 206. Motion is defined by a change in this proximity (so only changes in the z-axis, closer to or further away from the body). The capacitance is a function of proximity.

The structure can include a circuit that biases the feedback capacitor with a voltage proportional and inverted to the patient body voltage biasing the sensing capacitor. When a capacitor is biased by a voltage, and the proximity between the plates of the capacitor are modulated, a current is induced defined by i=dC/dt V. Because the circuit is continuously adapting the feedback capacitor bias voltage, and because the proximity of the sensing capacitor and the feedback capacitor are modulated by the same amount—the currents induced on each capacitor at “the first terminal” intrinsically cancel each other.

Referring to FIG. 2A, the exemplary device 200 can be configured with an active guard circuitry and shields 232, e.g, to degradation of the gain at large distances, e.g., from fringe capacitance that may form between the input Vin and output Vout of the amplifier.

FIG. 2B shows another example circuit diagram illustrating a sensing circuit design according to the principles described herein. As shown, a patient's skin and a first conductive plate form a capacitor (with a dielectric provided between the patient's skin and the conductive, capacitive sensing plate 206. The diagram also illustrates the guard 232. The feedback capacitor is shown representatively by two capacitive plates 208a and 208b and a skin surface foil 236. An amplifier 212 is shown, with a first terminal 240 coupled to the capacitive sensing plate/capacitive sensor 206 and a first plate of the feedback capacitor 208. The other terminal 244 of the amplifier 212 may be connected to ground when using a bipolar supply. FIG. 2C is a photograph of a prototype showing the capacitive contacts 204a, 204b, and 206.

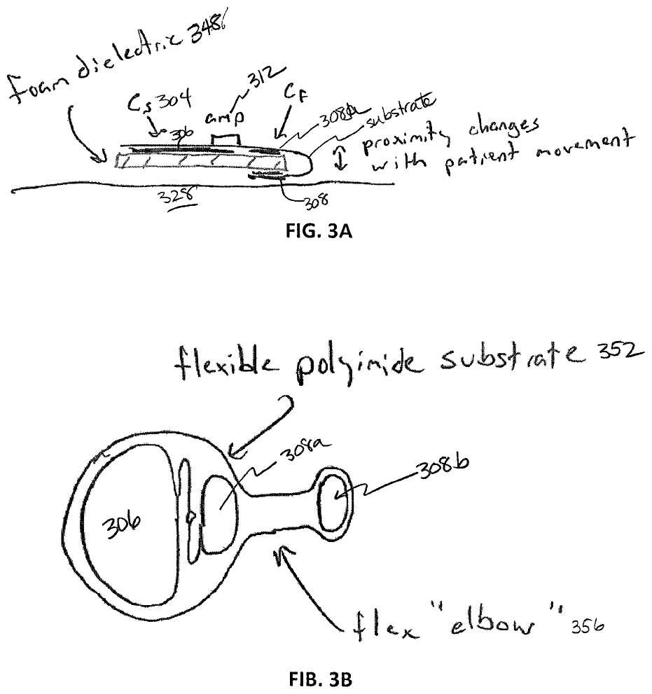

FIG. 3A shows an alternate design of a sensor according to the principles described herein. FIG. 3B shows an example construction of a sensor according to the principles described herein. The capacitance for the device of FIG. 3B can be determined as [symbol] . The gain can be determined as Cₛ/C_{f}, which, when substituted by the capacitance equation, provides a gain determined by Areaₛₑₙₛₑ/Area_{feedback}.

The example device of FIG. 3A includes a design in which one plate 308a of the feedback capacitor is tied to an input of the amplifier 312, is on the same side of a dielectric 348 as a plate 306 of the sensor capacitor 304 Cs that interacts with the patient's body 328 to form a capacitor with the dielectric 348, and is connected to a node common with the sensing capacitor plate 306. The other feedback capacitor plate 308b is on the opposite side of the dielectric 248 and is tied to the output of the amplifier 312. To facilitate use of the device, a common flexible substrate 352 may be used to host the capacitive sensing plate 306 and the plates 308a and 308b of the feedback capacitor. The flexible substrate may be folded/wrapped around the dielectric 348, which may be a compressible foam such that one plate 308a of the feedback capacitor 308 may be on the “skin side” of the dielectric 348, while the other feedback capacitor plate 308a is on the same side of the dielectric 348 as the capacitive sensing plate 306.

FIG. 3B shows an example of a polyimide substrate 352 having a capacitive plate 306 and both feedback plates 308a and 308b. As can be seen in FIG. 3B, the plates 306 and 308a share a common node, which the plate 308b is spaced apart so that it can be folded around a dielectric, e.g., via a flexible elbow 356.

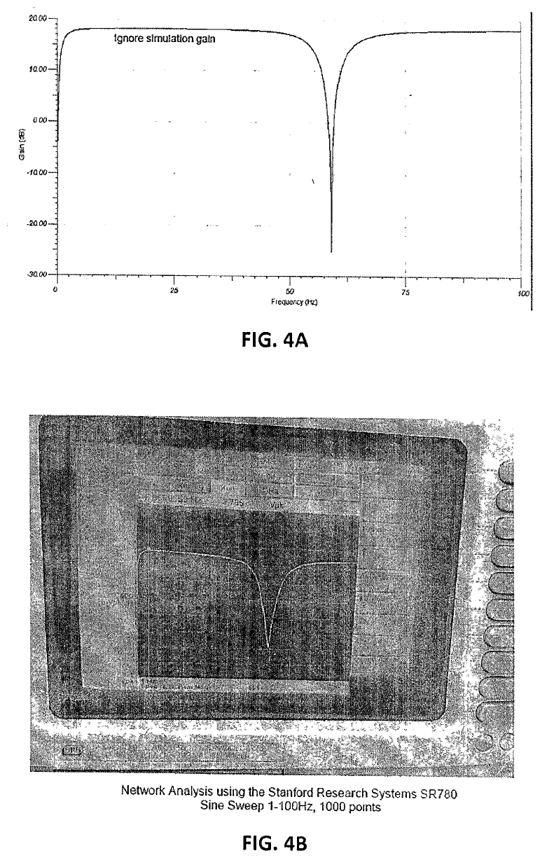

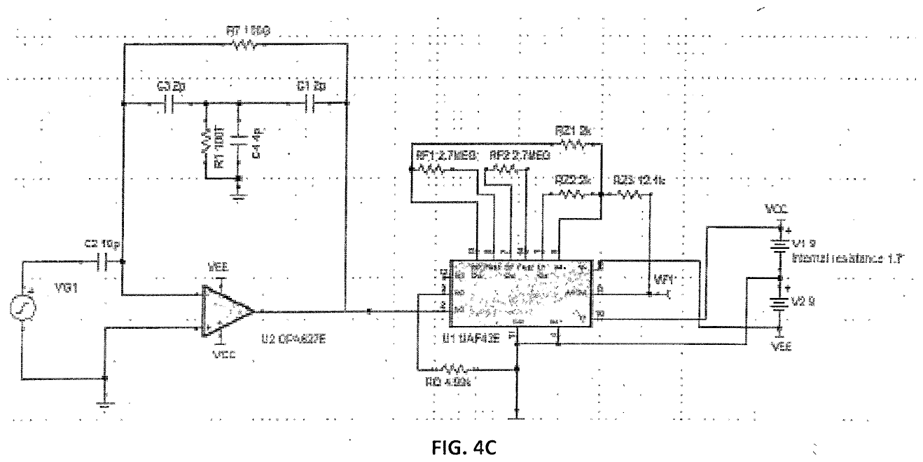

FIGS. 4A and 4B show simulations (FIG. 4A) and experiment results (FIG. 4B) of the prototype system. The simulation circuit is shown in FIG. 4C.

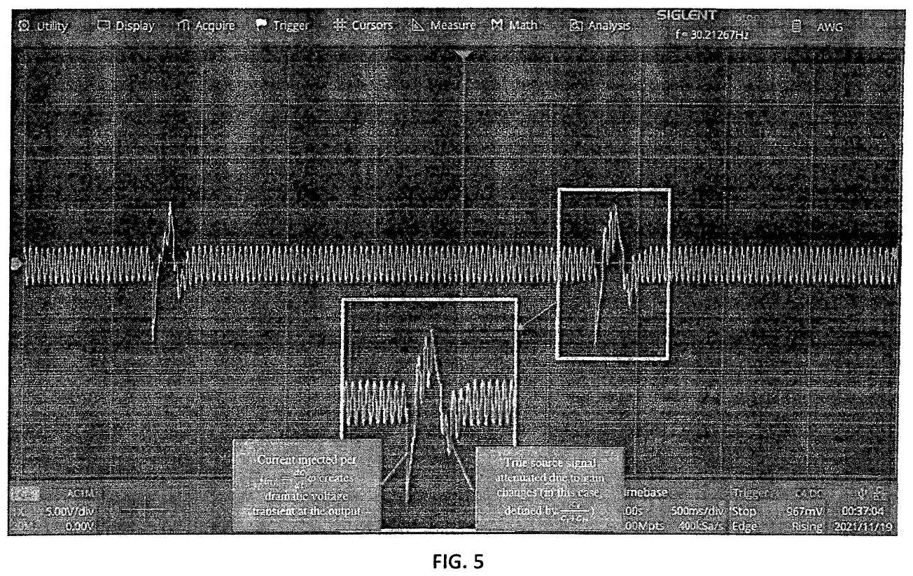

FIG. 5 shows an example motion artifact that may be picked up by the exemplary electrode device when only the sensing plate is employed. With respect to the outputs shown in FIG. 5, a large copper plane was excited with a 30Hx signal, and a capacitive sensor was placed 1 mm from the device with insulating foam in between. The sensor displacement was modulated +/−0.2 mm, producing the corruption (noise/unwanted current) pictured within the passband.

Indeed, the exemplary electrode device is intrinsically resilient to motion artifacts, consuming no extra computing or analog circuitry to implement as compared to typical pre-amplifier implementations. The exemplary electrode device can be particularly useful in being employed in new sensing environments that are highly noisy while providing low power performance (as well as non-noisy and conventionally used environments). It can facilitate long-term-wearable biosensing applications and can be manufactured with over-the-counter parts or custom (but cheap) circuit board designs, in contrast to the current state of the art, which requires specially designed and fabricated integrated circuits (or use as disposable or short-term applications and/or using expensive and complex circuitries).

Table 1 shows a comparison of the performance of the exemplary electrode device to other contact sensor designs.

TABLE 1

| | | | | | Proximity |
|---|---|---|---|---|---|
| | | | | | agnostic |
| | | | | | capacitive |
| | | | Pre-amplifier | Custom | feedback |
| | Capacitive | | with input | silicon unity | and DC path |
| | Feedback | Instrumentation | parasitic | pre-amp | according to |
| | with bias | amp with | cancellation | with input | principles |
| | current | manual capacitor | and custom | capacitance | described |
| Topology | compensation | network reset | guard | cancellation | herein |
| Gain at | 5.5 (100%) | 869 (100%) | 125 (100%) | ~1 (100%) | Not |
| 0.18 mm | | | | | measurable |
| Gain at 1 mm | 0.93 (17%) | 722 (83%) | 80 (64%) | 0.96 (98%) | 2.775 (92.5%) |
| Gain at 5 mm | 0.18(3%) | 193 (22%) | 19 (15%) | 0.791 (79.1%) | 1.34 (53.4%) |
| Loss (1 to 5) | 80% | 73% | 77% | 18% | 51.6% |

The following describes an example implementation of a standard non-contact biosensor. While motion is a significant concern, low coupling factor biosensors can still find use in applications with excellent patient adhesion. Applications for this style of biosensor could be in tightly bound mechanical wearables, such as watches or headbands.

### Prototype

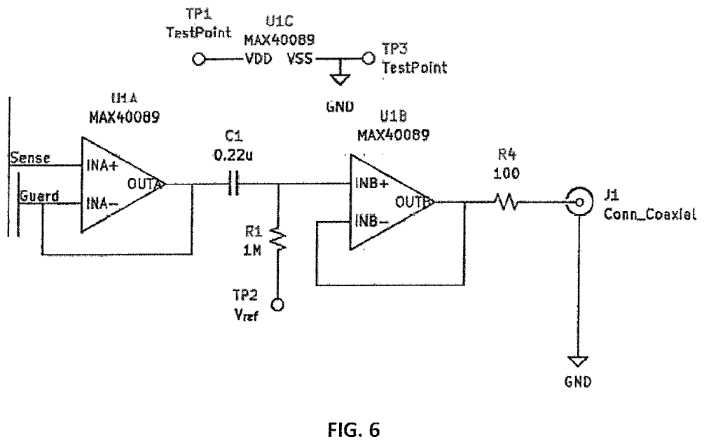

FIG. 6 is a schematic for an example capacitive electrode preamplifier according to the principles described herein. The prototype design discussed herein features the MAX40077 from Maxim Integrated, a dual amplifier package with suitable characteristics as described herein. The component is used to first buffer the sensing plate signal, then drive the coaxial cable, as shown in FIG. 6. Note the component label of MAX40089 in FIG. 6. This is a pin-compatible variant of the MAX40077. In the working design, the MAX40077 was used. One of skill in the art will appreciate that any appropriate amplifier circuit can be used in a device according to the principles described herein.

Since the operating point is defined by exploiting the anti-parallel diodes between the amplifier inputs, the DC-level is not well defined due to component variation. To combat this, the buffer pre-amplifier is high-pass filtered (sub-1 Hz corner) and fed to the second amplifier in the package to drive the cable. The MAX40077 has an input capacitance of 7 pF. In this design, a minimally acceptable coupling factor of 0.5 was used as a design target. Thus, the sensing plate is sized to be 7 pF at 1 mm.

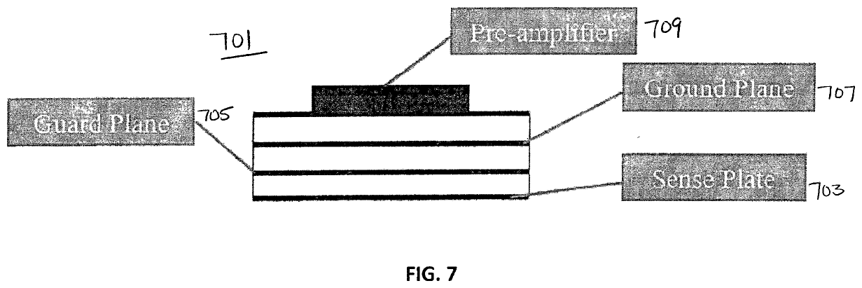

To minimize parasitics, the board-level design was implemented on a four-layer stack-up 701, as illustrated in FIG. 7. The capacitive level board-level stack up illustrated in FIG. 7 includes a sense plate 703, a guard plane 705, a ground plane 707, and a preamplifier 709. On the layer above the sensing plate, the guard node/plane, which includes the first amplifier output, is flooded to minimize ground-referred capacitance. Continuing up the stack-up, this is followed by a ground plane and then the active circuitry of the preamplifier design. This order takes inspiration from tri-axial cables used in precision ammeters.

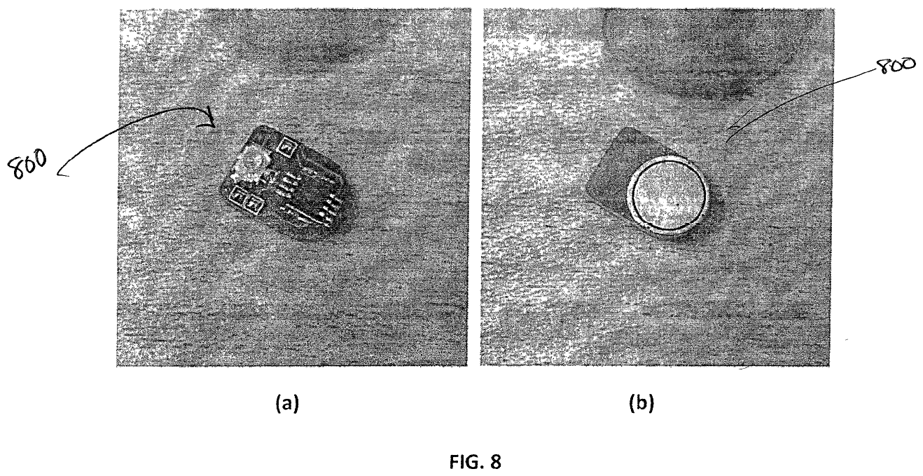

FIG. 8 is a photograph showing a prototype capacitive biosensing electrode 800 according to principles described herein-subpart (a) shows the preamplifier circuit on a first side of the device, and subpart (b) shows the sensing plate on a second side of the device. This design demonstrates a small, yet viable prototype capacitive biosensing electrode when compared to the literature, measuring in at roughly 8 mm by 12 mm.

As previously presented, the coupling factor of previous cap-mode biosensors degrades dramatically with motion. Similarly, in preamplifier designs with gain [7][8], where the gain is defined as a function of the feedback capacitor and the input (sense) capacitor, the performance may degrade because of motion. One could implement a system where an out-of-band signal is driven to the body and then picked off from the signal chain, with the goal of deriving the sense capacitance in real time. However, this adds complexity to the system and relies on a known good connection somewhere on the body.

However, these capacitive-type amplifier configurations, according to the principles described herein, hold an exciting characteristic. Their in-band gain is given by:

$$Vo = \frac{Cs}{Cf} Vl$$

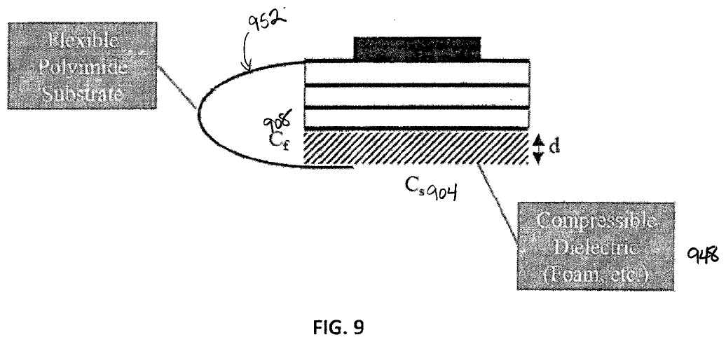

The sense capacitor is fabricated in process as a PCB thin foil layer fill coupled to the patient body. While the design is intrinsically weakened by the mechanical reality of the use case, this also provides the designer with the opportunity to control aspects of the design. Using rigid-flex polyimide fabrication processes, one can not only create the sensing capacitor in the fabrication process, but also the feedback capacitor. This is illustrated in FIG. 9. This mechanical structure comprising a flexible polyimide substrate 952 and a compressible dielectric foam 948 allows both the feedback capacitor 908 and sensing capacitor 904 to modulate with proximity. This mechanical tracking creates a proximity invariant gain, given as:

$$Vo = \frac{As}{Af} Vl$$

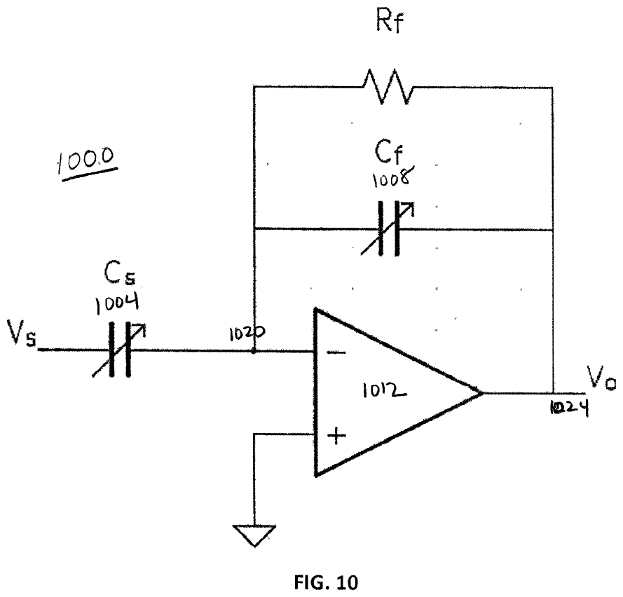

In mechanically constrained wearables, such as helmets, it is standard to have compressible pads, typically made from foam, to act as cushions for comfort. The described electrode can be integrated within pads, reducing motion artifact since displacement currents induced at the sense interface cancel each other out. Schematically, the simple circuit structure is shown in FIG. 10.

The exemplary electrode device can be configured with an active guard circuitry and shields, e.g., to degradation of the gain at large distances, e.g., from fringe capacitance that may be formed between the input and output of the amplifier.

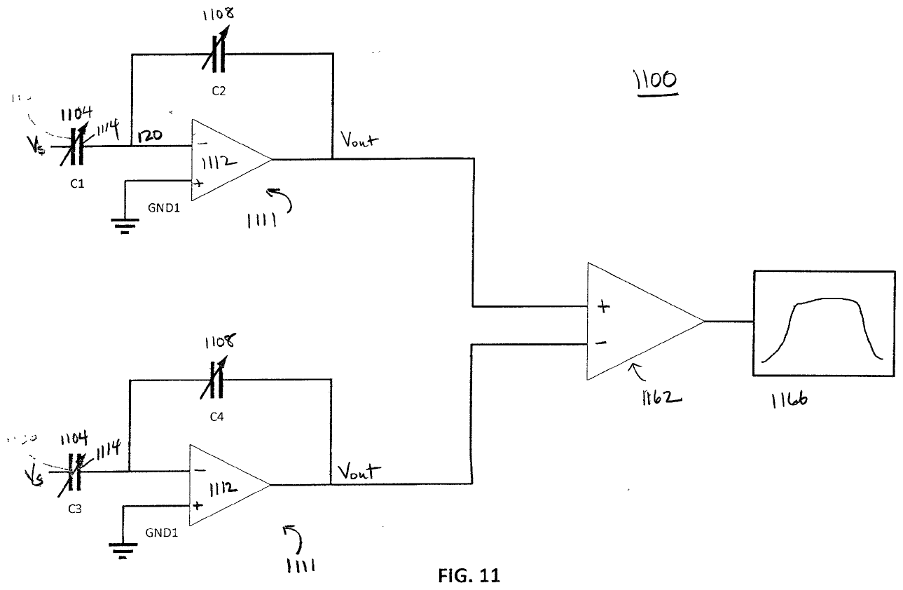

FIG. 11 illustrates an alternative circuit for implementation as a biosensor 1100 according to principles described herein, having two capacitive sensor circuits 1111 with feedback to compensate for motion. For example, each capacitive sensor circuit includes a capacitor plate 1114 that forms a sensing capacitor 1104 with the body (not shown) and a feedback capacitor 1108 where plates of feedback capacitor are located so that the feedback capacitor experiences the same motion as the sensing capacitor. Outputs Vout of each of the capacitive sensing circuits is provided to an instrumentation amplifier 1162, for example, with one output fed to an inverting terminal and one output fed to a non-inverting terminal of an amplifier 1162, the output of which may be fed to a bandpass filter 1166. The resulting output thus can be filtered to account for motion artifacts to produce a sensor result for various medical needs, such as ECG, EMG, or EEG measurements.

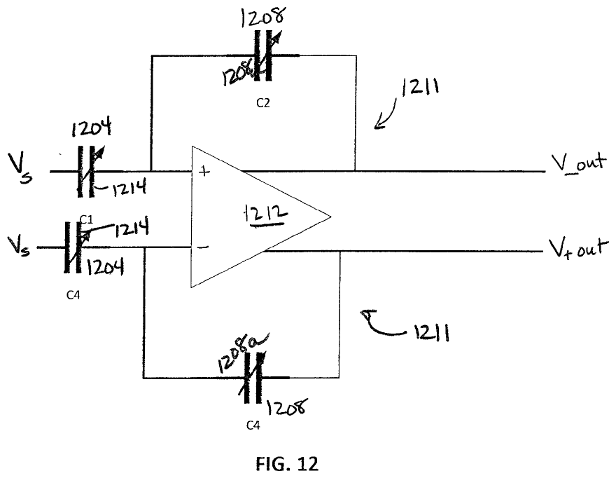

FIG. 12 illustrates another embodiment incorporating the basic sensing circuit described herein. The circuit of FIG. 12 includes two capacitive sensor circuits 1211 with feedback to compensate for the motion of a respective sensing capacitor 1204 (formed with the body). The capacitive sensing circuits share a single differential amplifier 1212 such that a plate 1214 of a first sensing capacitor 1204 and a plate 1208a of the feedback capacitor 1208 of a first circuit are input to a non-inverting terminal of the amplifier 1212, while a plate 1214 of a second sensing capacitor 1204 and a plate 1208a of the second feedback 1204 a capacitor are input to an inverting terminal of the differential amplifier 1212. The output of the differential amplifier is V₋ₒᵤₜ and V₊ₒᵤₜ, accordingly.

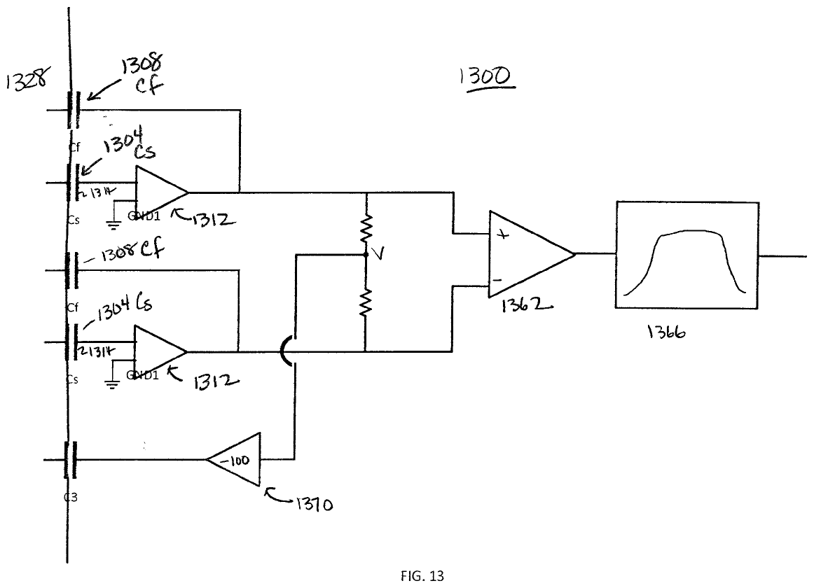

FIG. 13 illustrates another embodiment incorporating the basic biosensing circuit 1300 described herein, with improved common mode rejection (CMR). The circuit 1300 includes two capacitive sensor circuits 1311 with feedback to compensate for the motion of a respective sensing capacitor 1304 (formed with the body). For example, each capacitive sensor circuit includes a capacitor plate 1314 that forms a sensing capacitor 1304 with the body (not shown) and a feedback capacitor 1308 where plates of feedback capacitor are located so that the feedback capacitor experiences the same motion as the sensing capacitor. Outputs Vout of each of the capacitive sensing circuits is provided to an instrumentation amplifier 1162 and to a CMR boost amplifier 1370 also tied to a plate of a capacitor C3, forming a capacitor with the body 1328. For example, one output of the capacitive sensing circuits is fed to an inverting terminal while another output of the capacitive sensing circuit is fed to a non-inverting terminal of an amplifier 1362. Both outputs of the sensing circuits 1311 are coupled to the CMR boost amplifier 1370. The output of the instrumentation amplifier 1362 may be fed to a bandpass filter 1366.

A sensor device, according to principles described herein, may include an amplifier having a first input terminal, a second input terminal, and an output; a dielectric body; a capacitive sensor disposed on the dielectric body and having an output coupled to the first input terminal of the amplifier; a feedback capacitor having a first capacitive plate coupled to the output of the capacitive sensor and the first input terminal of the amplifier and having a second capacitive plate coupled to the output of the amplifier.

The dielectric body may include a compressible material, such as compressible foam. The dielectric body may include an adhesive.

The second input terminal of the amplifier may be tied to ground when using a bipolar supply. The second input terminal of the amplifier may be connected to a mid supply when using a unipolar supply.

The amplifier may actively bias the feedback capacitor with a negative potential to substantiate an equal but opposite charge to the first capacitive plate of the feedback capacitor connected to the input of the amplifier.

The feedback capacitor may be a variable capacitor.

The capacitive sensor may be formed by a first capacitive plate configured to comprise a sensing capacitor in combination with the dielectric body when in proximity with a body acting as a second capacitive plate.

The output of the amplifier may have a constant gain defined by capacitance Cs of the capacitive sensor and capacitance Cf of the feedback capacitor. The gain may be defined as Cs/Cf. Capacitance Cs of the capacitive sensor and capacitance Cf of the feedback capacitor may track together with the proximity of the capacitive sensor to a body acting as a second capacitive plate of the capacitive sensor.

The body may be skin. The skin may be human skin.

The first input terminal of the amplifier may be an inverting terminal, and the second input terminal of the amplifier may be a non-inverting terminal.

The sensor may further include a guard circuit protecting the capacitive sensor from fringe capacitance.

The sensor may be wearable. The sensor device is incorporated into a wearable device.

The sensor device may be employed for ECG, EMG or EEG measurements.

A biosensor according to principles described herein may include a flexible substrate; a first feedback capacitive plate on the flexible substrate; a second feedback capacitive plate on the flexible substrate; a sensor capacitive plate on the flexible substrate; and a biasing circuit connected between the second feedback capacitive plate, and the first feedback capacitive plate, wherein the first feedback capacitive plate is coupled to the sensor capacitive plate.

The biasing circuit may be an amplifier.

In an aspect, the flexible substrate may have a first side and a second side, with the first feedback capacitive plate on the first side of the flexible substrate; the second feedback capacitive plate is on the second side of the flexible substrate, and the sensor capacitive plate on the first side of the flexible substrate.

The flexible substrate may have a first side and a second side with the first feedback capacitive plate, the second feedback capacitive, and the sensor capacitive plate on the first side of the flexible substrate.

The biosensor may be wearable. The biosensor may be incorporated into a wearable device. The biosensor may include an adhesive on the flexible substrate.

The biosensor may be employed for ECG, EMG, or EEG measurements.

The flexible substrate may be made of polyimide.

The biosensor may include a foam dielectric on the sensor capacitive plate on a side opposite the flexible substrate.

Throughout this application, various publications may have been referenced. The disclosures of these publications in their entireties are hereby incorporated by reference into this application in order to more fully describe the state of the art to which this invention pertains. While it is reported in journal/conference papers of the use and design of non-contact designs, they do not address the issue of motion artifact, capacitor divider degradation, and other aforementioned technical implementation issues discussed herein.

An early non-contact design has been described in [1′]. Ref. [2′] describes a non-contact electrode that operates with an instrumentation amplifier. Ref. [3′] discloses a non-contact electrode that operates with discrete components with a custom guard circuit.

A more recent design employs a custom silicon pre-amp with input capacitance cancellation [4′], [5′]. While strapping input capacitance can help with a gain drop-off, it may not help in addressing injected current from changes in the sensing capacitor, as can be provided by the exemplary electrode design.

Refs. [6′]-[9′] discloses a non-contact electrode sensor with input bias current compensation. This is a mixed-domain data correction technique that can use immense amounts of power and is very large.

In contrast, the exemplary electrode device is configured with a structure that can intrinsically cancel unwanted induced currents, e.g., from motion, and maintains amplifier gain across a wide range of distances.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
[1′] A. Lopez and P. C. Richardson, “Capacitive Electrocardiogramd Bioelectric Electrodes,” in IEEE Transactions on Biomedical Engineering, vol. BME-16, no. 1, pp. 99-99, January 1969, doi: 10.1109/TBME.1969.4502613.
[2′] (also referre to herein as [2]) Y. M. Chi, S. R. Deiss and G. Cauwenberghs, “Non-contact Low Power EEG/ECG Electrode for High Density Wearable Biopotential Sensor Networks,” 2009 Sixth International Workshop on Wearable and Implantable Body Sensor Networks, 2009, pp. 246-250, doi: 10.1109/BSN.2009.52.
[3′] (also referred to herein as [3]) Y. M. Chi, S. R. Deiss and G. Cauwenberghs, “Non-contact Low Power EEG/ECG Electrode for High Density Wearable Biopotential Sensor Networks,” 2009 Sixth International Workshop on Wearable and Implantable Body Sensor Networks, 2009, pp. 246-250, doi: 10.1109/BSN.2009.52.
[4′] (also referred to herein as [4]) Y. M. Chi, C. Maier and G. Cauwenberghs, “Integrated ultra-high impedance front-end for non-contact biopotential sensing,” 2011 IEEE Biomedical Circuits and Systems Conference (BioCAS), 2011, pp. 456-459, doi: 10.1109/BioCAS.2011.6107826.
[5′] U.S. Pat. No. 9,360,501.
[6′] (also referred to herein as [1]) G. Peng and M. F. Bocko, “A low noise, non-contact capacitive cardiac sensor,” 2012 Annual International Conference of the IEEE Engineering in Medicine and Biology Society, 2012, pp. 4994-4997, doi: 10.1109/EMBC.2012.6347114.
[7′] G. Peng and M. F. Bocko, “Non-contact ECG employing signal compensation,” 2013 IEEE Biomedical Circuits and Systems Conference (BioCAS), 2013, pp. 57-60, doi: 10.1109/BioCAS.2013.6679639.
[8′] G. Peng, M. Sterling and M. Bocko, “Non-contact, capacitive biosensor electrodes for electrostatic charge reduction,” SENSORS, 2013 IEEE, 2013, pp. 1-4, doi: 10.1109/ICSENS.2013.6688199.
[9′] U.S. Pat. No. 9,037,221.
Medicine and Biology Society, 2012, pp. 4994 4997, doi: 10.1109/EMBC.
Conference, 2007, pp. 154 157, doi: 10.1109/BIOCAS.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

Throughout this application, various publications may have been referenced. The disclosures of these publications in their entireties are hereby incorporated by reference into this application in order to more fully describe the state of the art to which this invention pertains.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

1. A sensor device comprising:
an amplifier having a first input terminal, a second input terminal, and an output;
a dielectric body;
a capacitive sensor disposed on the dielectric body and having an output coupled to the first input terminal of the amplifier;
a feedback capacitor having a first capacitive plate coupled to the output of the capacitive sensor and the first input terminal of the amplifier and having a second capacitive plate coupled to the output of the amplifier.

2. The sensor device of claim 1, wherein the dielectric body comprises a compressible material, such as compressible foam.

3. The sensor device of claim 1, wherein the dielectric body comprises an adhesive.

4. The sensor device of claim 1, wherein the second input terminal of the amplifier is tied to ground when using a bipolar supply.

5. The sensor of claim 1, wherein the second input terminal of the amplifier is connected to a mid supply when using a unipolar supply.

6. The sensor device of claim 1, wherein the amplifier actively biases the feedback capacitor with a negative potential to substantiate an equal but opposite charge to the first capacitive plate of the feedback capacitor connected to the input of the amplifier.

7. The sensor device of claim 1, wherein the feedback capacitor is a variable capacitor.

8. The sensor device of claim 1, wherein the capacitive sensor is a first capacitive plate configured to comprise a sensing capacitor in combination with the dielectric body when in proximity with a body acting as a second capacitive plate.

9. The sensor device of claim 1, wherein output of the amplifier has a constant gain defined by capacitance Cs of the capacitive sensor and capacitance Cf of the feedback capacitor.

10. The sensor device of claim 9, wherein the gain is defined as Cs/Cf.

11. The sensor device of claim 1, wherein capacitance Cs of the capacitive sensor and capacitance Cf of the feedback capacitor track together with proximity of the capacitive sensor to a body acting as a second capacitive plate of the capacitive sensor.

12. The sensor device of claim 7, wherein the body is skin.

13. The sensor device of claim 12, wherein the skin is human skin.

14. The sensor device of claim 1, wherein the first input terminal of the amplifier is an inverting terminal, and the second input terminal of the amplifier is a non-inverting terminal.

15. The sensor device of claim 1, further comprising a guard circuit protecting the capacitive sensor from fringe capacitance.

16. The sensor device of claim 1, wherein the sensor is wearable.

17. The sensor device of claim 1, wherein the sensor device is incorporated into a wearable device.

18. The sensor device of claim 1, wherein the sensor device is employed for ECG, EMG or EEG measurements.

19. A biosensor, comprising;
a flexible substrate;
a first feedback capacitive plate on the flexible substrate;
a second feedback capacitive plate on the flexible substrate;
a sensor capacitive plate on the flexible substrate; and
a biasing circuit connected between the second feedback capacitive plate, and the first feedback capacitive plate, wherein the first feedback capacitive plate is coupled to the sensor capacitive plate.

20. The biosensor of claim 19, wherein the biasing circuit is an amplifier.

21.-29. (canceled)
